(19) United States
(12) Patent Application Publication
(10) Pub. No.: US 2025/0029721 A1
(43) Pub. Date: Jan. 23, 2025

(54) APPARATUS AND METHOD FOR PREDICTING TENDON RE-RUPTURE BASED ON ARTIFICIAL INTELLIGENCE

(71) Applicant: THE CATHOLIC UNIVERSITY OF KOREA INDUSTRY-ACADEMIC COOPERATION FOUNDATION, Seoul (KR)

(72) Inventors: Sung Hyun CHO, Seoul (KR); Yang Soo KIM, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/434,884

(22) Filed: Feb. 7, 2024

(30) Foreign Application Priority Data
Jul. 18, 2023 (KR) 10-2023-0092927

(51) Int. Cl. G16H 50/20; G06T 7/00

(52) U.S. Cl. CPC G16H 50/20; G06T 7/0012; G06T 2207/20081; G06T 2207/30004

(57) ABSTRACT

An apparatus and a method for predicting re-rupture of a tendon based an artificial intelligence (AI) are provided. The apparatus includes a communication module to make communication with an external device, an acquiring module to acquire at least one arthroscopic image including a surgical portion of a patient experiencing a surgery, a storage module to store at least one process based on the AI, and a control module to perform an operation for predicting the re-rupture of the tendon based on the AI, through the at least one process. The control module performs a pre-processing operation for the at least one arthroscopic image, predicts a probability of the re-rupture of the tendon by inputting the at least one arthroscopic image, which is pre-processed, into a pre-trained model based on the AI, and generates prediction information for the patient based on a prediction result.

### CROSS-REFERENCE TO RELATED APPLICATIONS

A claim for priority under 35 U.S.C. § 119 is made to Korean Patent Application No. 10-2023-0092927 filed on Jul. 18, 2023 in the Korean Intellectual Property Office, the entire contents of which are hereby incorporated by reference.

### BACKGROUND

Embodiments of the present disclosure described herein relate to an apparatus and a method for predicting the re-rupture of a tendon based on artificial intelligence (AI), and more particularly, relate to an apparatus and a method for predicting the re-rupture of a tendon based on artificial intelligence (AI), by using an arthroscopic image.

Arthroscopic cuff repair (ARCR) is one of main treatment options for the rupture of a rotator cuff, and patients and surgeons are increasingly interested in the outcomes from the ARCR.

Among them, re-rupture is one of the most important negative outcomes, and re-rupture has been reported as occurring in a certain percentage of patients during the past few decades.

As risk causes of the re-rupture of the tendon repaired after ARCR including the size of a large rupture in several studies are evaluated, risk factors for re-rupture of repaired tendons after ARCR, various factors including age and fat infiltration of rotator cuff have been extensively studied as risk factors, but contradictory results have been reported.

The fat infiltration by MRI indirectly reflects the quality of tendons of the rotator cuff and is an important risk factor for re-rupture. Surgeons may predict bad outcomes during tendon repair depending on the quality of the tendon, but there is no reliable way to evaluate or quantify the quality of the tendon quality until now.

Accordingly, a technology needs to be developed to easily predict the re-rupture of the tendon by analyzing the arthroscopic image using an artificial intelligence (AI)-based pre-trained model.

### SUMMARY

Embodiments of the present disclosure provide an apparatus and a method for predicting the re-rupture of a tendon based on artificial intelligence (AI), capable of pre-training a model based on a deep-learning algorithm using an arthroscopic image appropriate to determining the quality of the tendon, easily predicting the probability of the re-rupture for a surgical site of a relevant patient using a pre-trained model, such that a medical staff or a medical institution operates the relevant patient based on the probability of the re-rupture, thereby reducing the probability of the re-rupture.

Embodiments of the present disclosure provide an apparatus and a method for predicting the re-rupture of a tendon based on artificial intelligence (AI), capable of transmitting and providing prediction information, which is generated as the probability of the re-rupture is predicted based on an arthroscopic image of a relevant patient, to at least one relevant terminal which is preset (registered), such that the relevant patient is thoroughly cared after operation.

Problems to be solved by the present disclosure are not limited to the problems mentioned above, and other problems not mentioned will be clearly understood by those skilled in the art from the following description.

According to an embodiment, an apparatus for predicting re-rupture of a tendon based an artificial intelligence (AI) may include a communication module to make communication with an external device, an acquiring module to acquire at least one arthroscopic image including a surgical portion of a patient experiencing a surgery, a storage module to store at least one process based on the AI, and a control module to perform an operation for predicting the re-rupture of the tendon based on the AI, through the at least one process. The control module may perform a pre-processing operation for the at least one arthroscopic image, predict a probability of the re-rupture of the tendon by inputting the at least one arthroscopic image, which is pre-processed, into a pre-trained model based on the AI, and generate prediction information for the patient based on a prediction result.

Meanwhile, according to an embodiment a method for predicting re-rupture of a tendon based on artificial intelligence (AI), which is performed by an apparatus, may include acquiring at least one arthroscopic image including a surgical site of a patient experiencing a surgery, performing a pre-processing operation for the at least one arthroscopic image, predicting a probability of the re-rupture of the tendon by inputting the at least one arthroscopic image, which is pre-processed, into a pre-trained model based on the AI, and generating prediction information for the patient based on a prediction result.

In addition, a computer program stored in a computer-readable recording medium to execute a method to implement the present disclosure may be further provided.

In addition, a computer-readable recording medium for recording computer program to execute a method to implement the present disclosure may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a view illustrating a network structure of a system for predicting the re-rupture of a tendon based on AI, according to an embodiment of the present disclosure;

FIG. 2 is a view illustrating the configuration of an apparatus for predicting the re-rupture of a tendon based on AI, according to an embodiment of the present disclosure;

FIG. 3 is a view illustrating a method for predicting the re-rupture of a tendon based on AI, according to an embodiment of the present disclosure;

FIG. 4 is a view illustrating a method for training a pre-trained model based on AI, according to an embodiment of the present disclosure.

FIG. 5 is a view illustrating a series of operations for classifying and storing a plurality of arthroscopic images for each of different patients collected according to an embodiment of the present disclosure;

FIG. 6 is a view illustrating a series of operations of pre-processing a plurality of arthroscopic images for each of different patients collected according to an embodiment of the present disclosure;

FIGS. 7A and 7B are views illustrating arthroscopic images used for training the pre-trained model based on AI according to embodiments of the present disclosure;

FIG. 8 is a view illustrating a receiver operating descriptive curve during verification of a pre-trained model performed according to the present disclosure; and

FIGS. 9 and 10 are views illustrating the ROC curve and the confusion matrix obtained for each pre-trained model using the test set according to the present disclosure.

### DETAILED DESCRIPTION

Advantage points and features of the present disclosure and a method of accomplishing thereof will become apparent from the following description with reference to the following figures, wherein embodiments will be described in detail with reference to the accompanying drawings. However, the present disclosure may be embodied in various different forms, and should not be construed as being limited only to the illustrated embodiments. Rather, these embodiments are provided as examples so that the present disclosure will be thorough and complete, and will allow those skilled in the art to fully understand the scope of the present disclosure. The present disclosure may be defined by scope of the claims.

The terminology used herein is provided for explaining embodiments, but the present disclosure is not limited thereto. As used herein, the singular terms are intended to include the plural forms as well, unless the context clearly indicates otherwise. Furthermore, it will be further understood that the terms “comprises”, “comprising,” “includes” and/or “including”, when used herein, specify the presence of stated elements, steps, operations, and/or devices, but do not preclude the presence or addition of one or more other components, steps, operations and/or devices. The same reference numerals will be assigned to the same component throughout the whole specification, and “and/or” refers to that components described include not only individual components, but at least one combination of the components. It will be understood that, although the terms “first”, “second”, etc., may be used herein to describe various components, these components should not be limited by these terms. These terms are only used to distinguish one component from another component. Thus, a first component to be described below may be a second component without departing from the teachings of the present disclosure.

Unless otherwise defined, all terms (including technical and scientific terms) used herein have the same meaning as commonly understood by those skilled in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The same reference numerals will be assigned to the same components throughout the whole specification. In the following description of the present specification, all components are not described, and content well known in the art to which the present disclosure pertains or the duplication between embodiments will be omitted. The term “unit” or “module” used herein may refer to software or hardware such as field programmable gate array (FPGA) or application specific integrated circuit (ASIC), and the “unit” or “module” may perform some functions. The term “unit” or “module” is not limited to software or hardware. The term “unit” or “module” may be configured to be present in a storage medium to be assigned with addresses and may be configured to reproduce one or more processors. Accordingly, for example, the “unit” or “module” may include components, such as software components, object-oriented software components, class components, and task components, processes, functions, properties, procedures, subroutines, program code segments, drivers, firmware, micro-codes, circuits, data, database, data structures, tables, arrangements or variables. The components and the functions performed in the “unit” or “module” may be formed by combining the smaller number of components and the “unit” or the “module”, or more divided into additional components and the “unit” or the “module”.

In the whole specification, when a certain part is “linked to”, “coupled to”, or “connected with” another part, the certain part may be directly linked to, coupled to or connected with the another part, and an indirection link, an indirection coupling, or an indirection connection includes a link, a coupling, or a connection through a wireless communication network.

It will be understood that the terms “comprises,” “comprising,” “includes” and/or “including,” when used herein, specify the presence of stated elements and/or components, but do not preclude the presence or addition of one or more other elements and/or components.

In the present specification, when a member is positioned on another member, this includes not only when the member is in contact with the other member, but also when another member is present between the two members.

In the specification, the term “first and/or second” will be used to distinguish between components, and the components are not limited to the above-described terminology.

The articles “a”, “an,” and “the” are singular in that they have a single referent, but the use of the singular form in the specification should not preclude the presence of more than one referent.

Reference numerals in steps are only for the illustrative purpose, and not used to describe the sequence of the steps. The steps may be replicated in a sequence different from a sequence, which is described, unless otherwise specified.

Hereinafter, the terminology of the present disclosure will be defined.

According to the present disclosure, the Ai-based pre-trained model is a deep-learning based prediction model. Through the pre-trained model or the prediction model, the probability of the re-rupture for a surgical site of a relevant patient may be predicted and prediction information may be generated. In this case, the deep-learning scheme is not limited to any specific scheme, but may employ at least one scheme depending on situations. In this case, an AI algorithm may include a recurrent neural network (RNN) or a transformer, but the present disclosure is not limited thereto. For example, the AI algorithms may include various AI algorithms.

In the present disclosure, although a predicting device 100 is described by way of example, the predicting device 100, which serves as a device to generate and provide prediction information by predicting the re-rupture of the tendon of a relevant patient experienced ARCR based on the AI, may include various devices to perform arithmetic processing. In other words, the predicting device 100 may further include a server, a computer, and/or a portable terminal or may be in any one form thereof, but the present disclosure is not limited thereto.

In this case, the computer may include, for example, a notebook equipped with a web browser, a desktop, a laptop, a tablet PC, or a slate PC.

The server is a server that processes information by communicating with an external device, and may include an application server, a computing server, a database server, a file server, a game server, a mail server, a proxy server, and a web server.

The portable terminal, which is, for example, a wireless communication device that guarantees portability and mobility, may include all kinds of handheld-based wireless communication devices, such as a personal communication system (PCS), a global system for mobile communications (GSM), a personal digital cellular (PDC), a personal handphone system (PHS), a personal digital assistant (PDA), International Mobile Telecommunication (IMT)-2000, A code division multiple access (CDMA), (W-Code Division Multiple Access (W-CDMA), wireless broadband Internet terminal (WiBro), smart phone, and wearable devices, such as a watch, a ring, a bracelet, an anklet, a necklace, glasses, contact lenses, or a head-mounted device (HMD).

The ‘arthroscopic image’ is image data photographed to include a surgical site before, during, and after the operation of the corresponding patient who performed ARCR, and may be obtained through X-ray, Computer tomography (CT), Magnetic resonance imaging (MRI), a camera, etc., and is used to predict prognosis such as re-rupture. The arthroscopic image may be collected from a separate imaging device as well as other devices possessed or equipped by various management institutions, managers, medical staff, and medical institutions. In this case, the arthroscopic image has a form of time-series data collected for each patient during a predetermined period.

First, the present disclosure aims to generate and provide prediction information by photographing the surgical site of the patient during operation and predicting the probability of re-rupture of the tendon in real time, allowing the medical staff to consider the prediction information during surgery, and to transmit and provide the prediction information to at least one preset (registered) relevant terminal after surgery, such that the relevant patient is thoroughly cared after operation.

Hereinafter, an operating principle and embodiments of the present disclosure will be described with reference to the accompanying drawings.

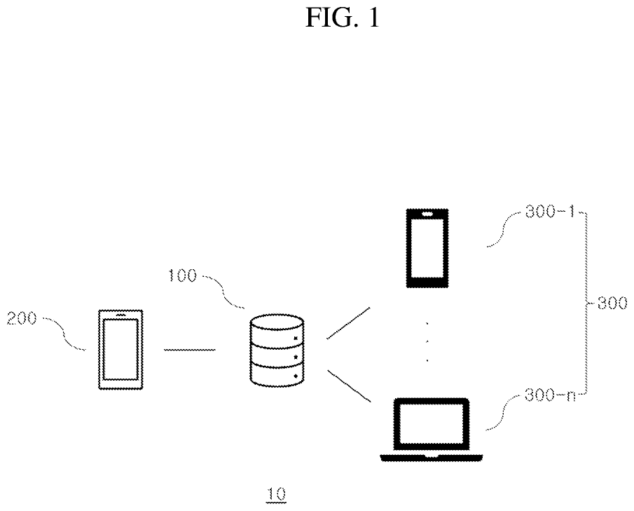

FIG. 1 is a view illustrating a network structure of a system for predicting the re-rupture of a tendon based on AI, according to an embodiment of the present disclosure.

Referring to FIG. 1, a system (hereinafter, a predicting system) 10 for predicting the re-rupture of the tendon based on AI, according to an embodiment of the present disclosure may be configured to include the predicting device 100, a managing terminal 200, and at least one user terminal 300. In this case, although only one managing terminal 200 is illustrated in FIG. 1, at least one managing terminal 200 may be provided like the at least one user terminal 300. Accordingly, the type and the number of the managing terminal 200 are not limited.

First, the predicting device 100 may predict the probability of the re-rupture of the tendon by collecting patient information including the personal information and/or an arthroscopic image of a patient based on a storage module 150, a separate photographing device, and a separate database, preprocessing the arthroscopic images, and inputting the pre-trained model based on AI. Accordingly, the predicting device 100 may generate prediction information for a relevant patient based on the prediction result and provide the prediction information by transmitting the prediction information to the preset (registered) managing terminal 200 and the at least one user terminal 300.

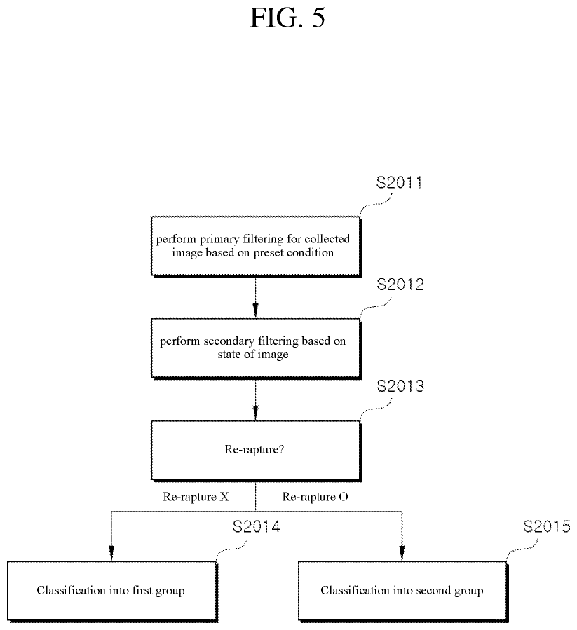

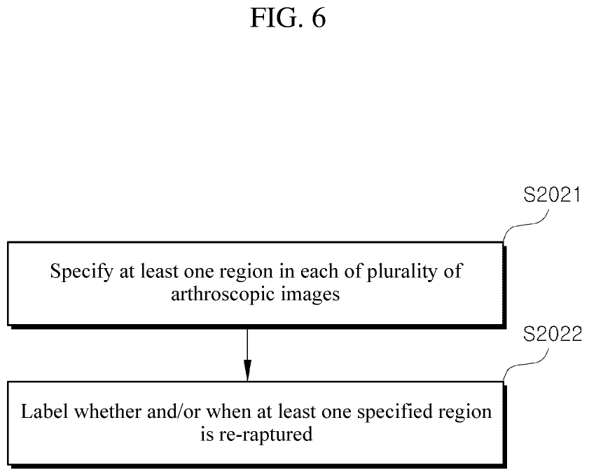

To this end, the predicting device 100 may implement a pre-trained model by collecting the patient information on at least one different patient and performing a relevant training operation on an original model. The detailed operation of implementing the pre-trained model will be described with reference to FIGS. 5 and 6.

Meanwhile, the managing terminal 200 is a terminal belonging to a medical staff, a medical institution, a manager, a managing institution recording or managing medical information including an arthroscopic image in addition to personal information on each of at least one patient, in which the personal information and the medical information are collectively referred to ‘patient information’). The managing terminal 200 may determine whether to provide the patient information in response to the request of the predicting device 100, and may provide the patient information to various organizations in addition to the predicting device 100.

In addition, the managing terminal 200 may receive the prediction information generated by the predicting device 100 based on the patient information of the relevant patient, may receive the prediction information in real time when performing the operation of the relevant patient, and may perform an operation based on the prediction information.

Meanwhile, the at least one user terminal 300 is a terminal belonging to the relevant patient and at least one guardian, which is preset, corresponding to the patient. The at least one user terminal 300 may refer to a terminal of a user allowed to receive specific information from the predicting device 100. In other words, the at least one user terminal 300 is a concept including a patient terminal and a guardian terminal. The at least one user terminal 300 may be previously registered to approach the predicting device 100 or approach the predicting device 100 through the managing terminal 200.

Meanwhile, the managing terminal 200 and the user terminal 300 may be a computer, an ultra-mobile PC (UMPC), a workstation, a netbook, personal digital assistants (PDA), a portable computer, a wireless phone, a mobile phone, a smart phone, a pad, a smart watch, a wearable device, a wearable device, a wearable device, a smart phone, a smart watch, an e-book, a portable multimedia player (PMP), a navigation device, a black box or a digital camera, or other mobile communication terminals allowing a medical staff (including a medical institution, a manager, or a managing institution) and/or a user (including a patient or a guardian for the patient) to install and execute desired multiple application programs (that is, applications). In other words, the managing terminal 200 and the at least one user terminal 300 may be provided in various forms, but the present disclosure is not limited thereto.

Meanwhile, the managing terminal 200 and the at least one user terminal 300 represent at least one preset relevant terminal, and a person, which is registered (set) by an authorized concerned person in addition to the medical staff (including the medical institution, the manager, and the managing institution) and/or a user (including a patient and a guardian) described above, may receive the prediction information.

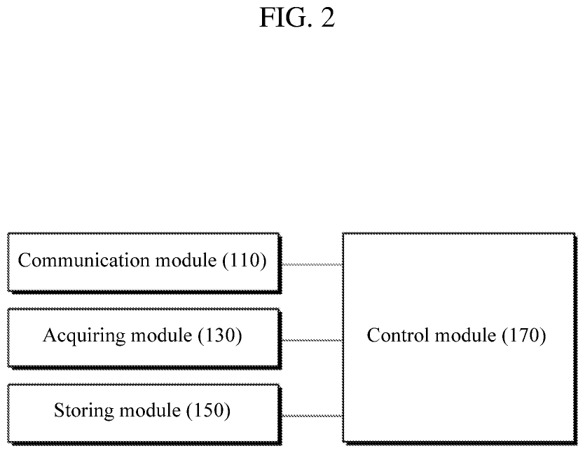

FIG. 2 is a view illustrating the configuration of an apparatus for predicting the re-rupture of a tendon based on AI, according to an embodiment of the present disclosure.

Referring to FIG. 2, the apparatus (hereinafter, the predicting device) 100 for predicting the re-rupture of the tendon based on AI, according to an embodiment of the present disclosure may include a communication module 110, an acquiring module 130, the storage module 150, and a control module 170.

The communication module 110 may transmit or receive at least one piece of information or data together with at least one device/terminal. In this case, the at least one device/terminal may be a device/terminal to receive prediction information based on AI, or a device/terminal to provide various types of data/information allowing the generation of the prediction information, and the type and the shape of the device/terminal is not limited thereto.

In addition, the communication module 110, which makes communication with other devices, allows wireless signals to be transmitted and received in a communication network based on a wireless Internet technology.

The wireless Internet technology includes a wireless LAN (WLAN), Wireless-Fidelity (Wi-Fi), Wireless Fidelity (Wi-Fi) Direct, Digital Living Network Alliance (DLNA), Wireless Broadband (WiBro), World Interoperability for Microwave Access (WiMAX), High Speed Downlink Packet Access (HSDPA), High Speed Uplink Packet Access (HSUPA), Long Term Evolution (LTE), LTE-A (Long Term Evolution-Advanced), and the predicting device 100 transmits and receives data according to at least one wireless Internet technology in a range including Internet technology not listed above.

Short range communication may be supported by using at least one of Bluetooth™, Radio Frequency Identification (RFID), Infrared Data Association (IrDA), Ultra Wideband (UWB), ZigBee, Near Field Communication (NFC), Wireless-Fidelity (Wi-Fi), Wi-Fi Direct, and Wireless Universal Serial Bus (Wireless USB) technologies. The wireless area networks may support wireless communication between the predicting device 100 and the managing terminal 200 and/or the at least one user terminal 300. In this case, the short range wireless communication network may be a wireless personal area network.

The acquiring module 130 is inserted into the body of the patient under an operation to acquire at least one arthroscopic image including the surgical site (the surgical target site). The acquired arthroscopic image may be stored as one pieces of patient information in the storage module 150 to be described below or an additional database.

The storage module 150 may store data on at least one process (algorithm) for generating prediction information based on AI or program obtained by reproducing the process. In addition, the storage module 150 may further store a process to perform other operations, and the present disclosure is not limited thereto.

The storage module 150 may store various types of data for supporting various functions of the predicting device 100 as well as the patient information (including the personal information and the arthroscopic image of each patient) on at least one patient. The storage module 150 may store data or instructions for multiple application programs (or applications) driven in the predicting device 100 or the operations of the predicting device 100. At least a portion of the application program may be downloaded from an external server through wireless communication. Meanwhile, the application program may be stored in at least one memory provided in the storage module 150, installed in the predicting device 100, and driven to execute an operation (or a function) by at least one processor stored in the storage module 150 through the control module 170.

Meanwhile, at least one memory may include at least one of a flash memory type, a hard disk type, a multimedia card micro type, a card type memory (e.g., SD or XD memory), a random access memory (RAM), a static random access memory (SRAM), a read-only memory (ROM), an electrically erasable read-only memory (EPROM), a programmable read-only memory (PROM), a magnetic disk, or an optical disk. In addition, the memory may store information temporarily, permanently, or semi-permanently, and may be provided in a built-in or detachable manner.

In addition, the storage module 150 may be implemented in the form of a database that stores various types of information necessary to generate artificial intelligence-based prediction information or may be linked with a separate external server.

Meanwhile, the control module 170 may perform an operation related to the application program, may control all components in the predicting device 100 through at least one processor to process an input signal, an output signal, data, or information or to execute an instruction, an algorithm, and an application program stored in at least one memory such that various processes are performed, and may provide or process information or a function appropriate to generating the prediction information based on AI.

In details, the control module 170 performs pre-processing on at least one arthroscopic image including the surgical site of the patient under operation, which is obtained through the acquiring module 130, inputs the preprocessed at least one arthroscopic image, into the pre-trained model based on AI to predict the probability of re-rupture of the tendon on the surgical site, and generates prediction information for the patient based on the prediction result. In addition, the control module 170 may transmit and provide the generated prediction information to at least one of the managing terminal 200, which is previously registered or set, or the at least one user terminal 300.

To this end, the control module 170 may form learning data and pre-train the original model based on the formed learning data. In this case, the control module 170 may collect and pre-process a plurality of arthroscopic images for each of different patients for a preset period, and form the preprocessed arthroscopic images as learning data. Thereafter, a pre-trained model may be implemented (built) by inputting the learning data to the original model to train the original model.

In this case, the control module 170 may remove at least one layer from the original model, finely adjust the parameter using the additional layer, and perform the training operation in a preset number of times using average square root propagation (RMSProp) at a preset speed.

Meanwhile, when pre-processing the plurality of arthroscopic images, the control module 170 specifies at least one region in each of the plurality of arthroscopic images, and labels at least one of whether the specified region is re-ruptured or a time point at which the specified region is re-ruptured, to each of the at least one area specified above. In this case, various pieces of medical information included in the patient information of different patients may be used for the labeling.

In addition, the control module 170 may perform an operation of evaluating and verifying the performance of the pre-trained model using predictive accuracy, F1 score, AUC, sensitivity, and specificity.

For example, the following <Equation 1> may be used to calculate the prediction accuracy and F1 score, and the Youden J value may be used to calculate the threshold value for the sensitivity and the threshold for the specificity.

$$Predictive accuracy = \frac{TP + TN}{TP + FP + FN + TN} \qquad \underline{Equation 1}$$

$$Positive predictive value = \frac{TP}{TP + FP}$$

$$Negative predictive value = \frac{TN}{TN + FN}$$

$$F 1 score = 2 \times \frac{( Senstivity \times Positive predictive value )}{( Senstivity + Positive predictive value}$$

In this case, ‘TP’ denotes ‘true positive’, ‘TN’ denotes ‘true negative’, ‘FP’ denotes ‘false positive’, and ‘FN’ denotes ‘false negative’.

Accordingly, the control module 170 may predict the probability of re-rupture of the tendon on the corresponding surgical site, based on the arthroscopic image of the relevant patient, and generate prediction information as the prediction result. In this case, the surgical site indicates a part corresponding to each joint of the body, and may include a shoulder joint, an elbow joint, a finger joint, a hip joint, a knee joint, a foot joint, and a spine joint. In addition, the prediction information may include a re-rupture prediction region based on at least one region specified in the arthroscopic image including the surgical site of the relevant patient, and a tendon state, a probability of the re-rupture of the tendon, and a re-rupture prediction time for each region.

In addition, the control module 170 may perform the categorization depending on the probability of the re-rupture of the patient

As described above, although the control module 170 performs the pre-training operation, the predicting device 100 may include a separate training module (not illustrated) to perform the pre-training operation.

In addition, the detailed operation of the control module 170 will be described based on the respective drawings below.

Meanwhile, although not illustrated in FIG. 2, the predicting device 100 may further include a display module to display and provide prediction information or may be connected to a separate display device through wired communication or wireless communication, such that a medical staff performing an operation easily recognizes the prediction information in real time. In this case, the separate display device may be included in the managing terminal 200 described above.

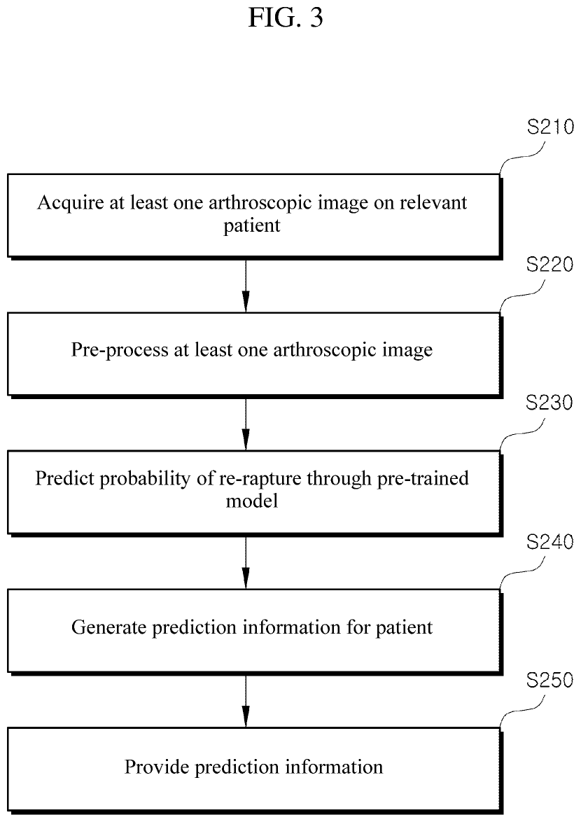

FIG. 3 is a view illustrating a method for predicting the re-rupture of the tendon based on AI, according to an embodiment of the present disclosure.

Referring to FIG. 3, the predicting device 100 acquires at least one arthroscopic image including the surgical site of the patient experiencing the operation (S210), and pre-processes the at least one arthroscopic image (S220).

Next, the predicting device 100 inputs the at least one arthroscopic image pre-processed in S220 into a pre-trained model based on AI to predict the probability of re-rupture of the tendon at the surgical site (S230), and generates prediction information for the patient based on the prediction result (S240).

Thereafter, the predicting device 100 transmits and provides the prediction information generated in S240 to at least one of the managing terminal 200, which is previously registered (set), or the at least one user terminal 300 (S250)

In this case, S250 is not an essential operation, but may be set to be automatically or manually performed depending on the settings of the predicting device 100, by the user or the manager. In other words, S250 may be omitted.

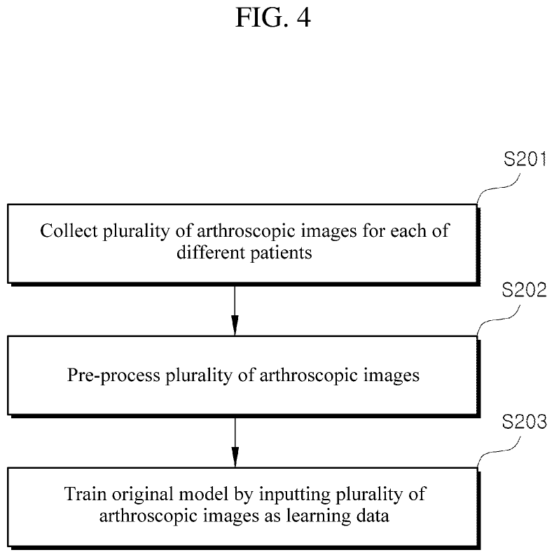

FIG. 4 is a view illustrating a method for training a pre-trained model based on AI according to an embodiment of the present disclosure.

Referring to FIG. 4, the predicting device 100 collects a plurality of arthroscopic images for each of the different patients during a preset period (S201), and pre-processes the collected arthroscopic images (S202). In this case, the plurality of arthroscopic images include various images captured during the preset period for each of the different patients in order of time series. Meanwhile, when pre-processing, the personal information (including an age, a gender, a weight, and a height) and medical information (including a medical history, an operation history, and a prescription history,) on each patient corresponding to each arthroscopic image may be further used, and the personal information and medical information may be collected through the storage module 150 of the predicting device 100 or a separate database.

Next, the predicting device 100 constructs the plurality of pre-processed arthroscopic images as learning data, inputs the learning data to the original model, and trains the original model (S203).

FIG. 5 is a view illustrating a series of operations for classifying and storing a plurality of arthroscopic images for each of different patients collected according to an embodiment of the present disclosure, and is a view specifically illustrating S201 of FIG. 3.

Referring to FIG. 5, the predicting device 100 primarily filters the plurality of arthroscopic images based on the preset conditions (S2011). For example, the preset conditions may be limited to an inappropriate follow-up period, tendon transfer or allograft augmentation, the timing of re-rupture (e.g., re-rupture 2 years after the surgery), a surgical site (e.g., suture of the lower scapular muscle of biceps), which may be set and changed by a manager, or a user of the predicting device 100.

Next, the predicting device 100 performs secondary filtering based on the states of a plurality of arthroscopic images which are primarily filtered (S2012). For example, image quality below a preset threshold or a broken image is excluded.

Next, the predicting device 100 determines whether a patient has re-rupture based on each of the plurality of arthroscopic images that have been secondarily filtered (S2013), classifies the image of the patient having no re-rupture into a first group to be managed depending on the determination result (S2014), and classifies the patient having re-rupture a second group to be managed depending on the determination result (S2015).

FIG. 6 is a view illustrating a series of operations of pre-processing a plurality of arthroscopic images for each of different patients collected according to an embodiment of the present disclosure, and is a view specifically illustrating S202 of FIG. 3.

Referring to FIG. 6, the predicting device 100 specifies at least one region in each of the at least one arthroscopic image (S2021), and labels whether and/or when the at least one specified region is ruptured (S2022).

In the above-described S2021, the predicting device 100 needs to specify a segmented part. Accordingly, the predicting device 100 specifies the segmented part. For example, the predicting device 100 may specify various regions, like specifying the surface having the tendon.

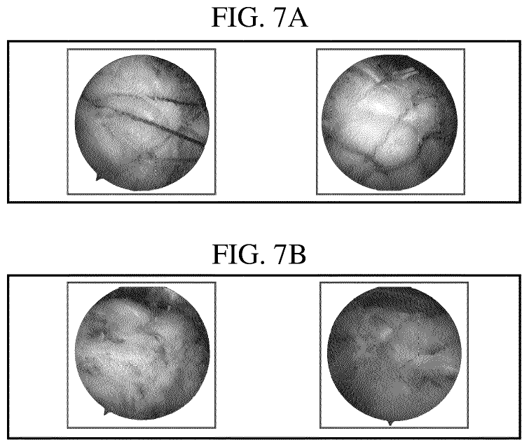

FIGS. 7A and 7B are views illustrating arthroscopic images used for training the pre-trained model based on AI according to embodiments of the present disclosure.

FIG. 7A represents an arthroscopic image taken immediately after the operation of a patient having no re-rupture and an arthroscopic image taken after a certain period of time from the operation of the patient. The arthroscopic images may be classified into the first group to be managed while serving as time-series data of the patient.

FIG. 7B represents an arthroscopic image taken immediately after the operation of a patient having re-rupture, and an arthroscopic image taken after a certain period of time from the operation of the patient. The arthroscopic images may be classified into the second group to be managed while serving as time-series data of the patient.

Meanwhile, although FIGS. 7A and 7B illustrate two arthroscopic images of the patient, but the present disclosure is not limited to the number of stored arthroscopic images. For example, a plurality of arthroscopic images taken repeatedly in a short period of time may be used.

Hereinafter, the results of performance tests through arthroscopic images of various patients will be described based on the pre-trained model implemented according to the present disclosure.

The following <Table 1> illustrates demographic data of study participants. After a total of 1,394 arthroscopic images stored in a medical image storage and transmission system (PACS) were acquired from 580 patients, the first group (the patient group having no re-rupture) and the second group (the patient group having re-rupture) included 1,138 and 256 images acquired from 514 and 66 patients, respectively. The average age of the study participants was 61.59 years in the first group and 64.92 years in the second group, which made a meaningful difference between the two groups (p<0.01). The rupture sizes in front and rear portions and inner and outer portions of supraspinatus were significantly larger in the second group (p<0.01)

TABLE 1

| | Non-retear group | Retear group | |
|---|---|---|---|
| | (n = 514) | (n = 66) | P-value |
| Number of arthroscopic | 1,138 | 256 | |
| images | | | |
| Sex, males:females | 181:333 | 28:38 | 0.64 |
| Age, years | 61.59 ± 8.64 | 64.92 ± 6.67 | <0.01 |
| Involved arm, right:left | 352:162 | 49:17 | 0.42 |
| SSP tear size (anterior to | 1.18 ± 0.72 | 2.25 ± 0.93 | <0.01 |
| posterior), cm | | | |
| SSP tear size (medial to | 1.29 ± 0.85 | 2.60 ± 0.95 | <0.01 |
| lateral retraction), cm | | | |
| Full thickness:Partial | 302:212 | 63:3 | <0.01 |
| thickness | | | |

Meanwhile, following Table 2 illustrates the cross-validation accuracy score for each CNN classifier model as a multiple, and the accuracy of the model for the test set was compared. It may be determined that the accuracy is improved, as the number of folds is increased. The average validation accuracies of VGG16 and Xception were 83% and 89%, respectively, and the average validation accuracies were the highest in DenseNet as 91%. In addition, the average validation AUC values were 0.78±0.02, 0.86±0.16, and 0.91±0.11, respectively, so VGG16, Xception, and DenseNet showed the highest values, while DenseNet showed the best AUC values in layered verification.

TABLE 2

| | Fold 1 | Fold 2 | Fold 3 | Average | Test |
|---|---|---|---|---|---|
| VGG16 | 0.83 | 0.84 | 0.83 | 0.83 ± 0.06 | 0.76 |
| DenseNet | 0.80 | 0.94 | 0.99 | 0.91 ± 0.10 | 0.91 |
| Xception | 0.80 | 0.91 | 0.95 | 0.89 ± 0.08 | 0.87 |

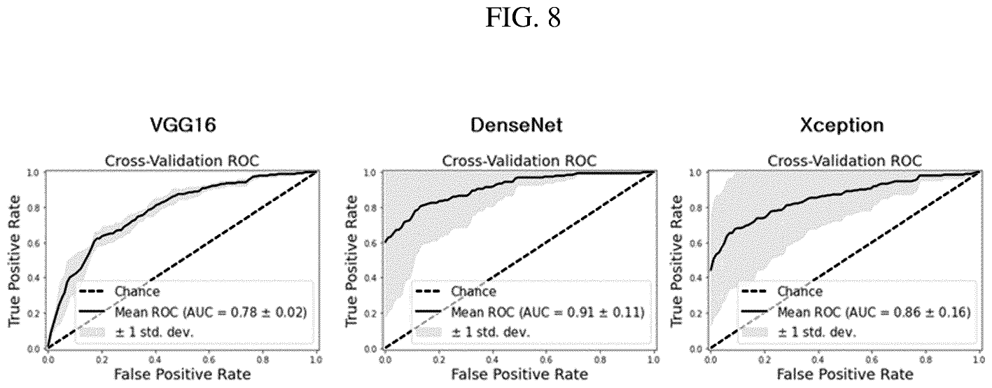

FIG. 8 is a view illustrating a receiver operating descriptive curve during verification of a pre-trained model performed according to the present disclosure.

Referring to FIG. 8, it may be recognized that all three models show the accuracy of 80% to 90%.

The performance of each classifier model for this test set is summarized in Table 3 below. The test set was not included in the training-verification procedure, and the accuracy value of the test set was lower than the average verification value (see Table 2 above). The accuracies of VGG16, DenseNet, and Xception were 76%, 91%, and 87%, respectively. It may be recognized that the AUC values of the CNN classifier using the three models all exceeded 0.8, and the AUC values of the VGG16 and Xception models for the test set were 0.83 and 0.91, respectively. In terms of model performance, DenseNet showed the highest AUC value (0.92).

TABLE 3

| | VGG16 | DenseNet | Xception |
|---|---|---|---|
| AUC | 0.83 | 0.92 | 0.91 |
| Accuracy | 0.76 | 0.91 | 0.87 |
| F1-score | 0.78 | 0.91 | 0.88 |
| Sensitivity | 0.80 | 0.84 | 0.84 |
| Specificity | 0.70 | 0.93 | 0.89 |

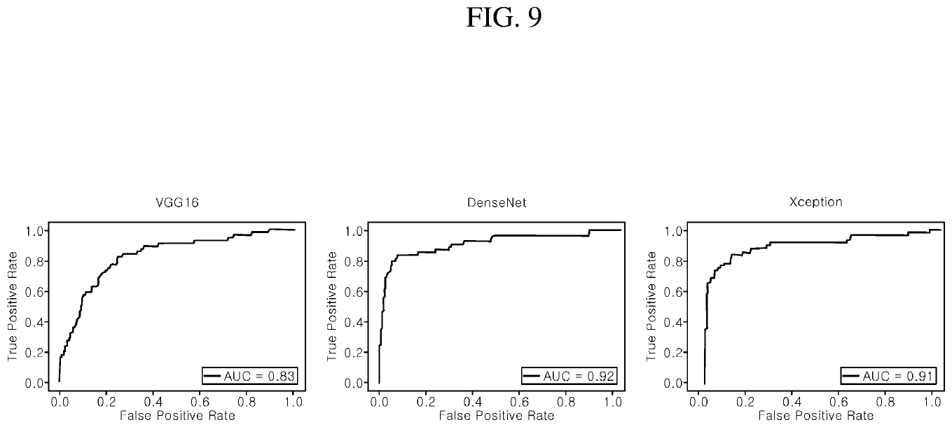

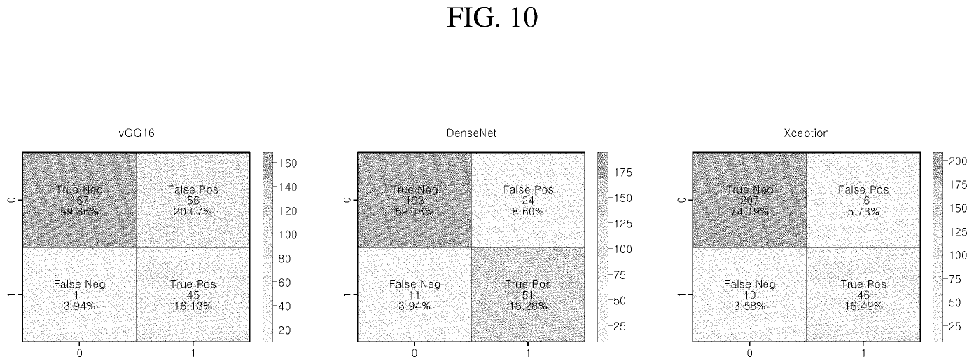

FIGS. 9 and 10 are views illustrating an ROC curve and the confusion matrix obtained for each pre-trained model using the test set according to the present disclosure.

In particular, the confusion matrix of DenseNet in FIG. 10 illustrates that the classifier accurately predicted the re-rupture with an accuracy of more than 90% in arthroscopic images during surgery.

Consequently, it could be confirmed that the CNN classifier algorithm according to the present disclosure predicts degeneration with higher accuracy based on arthroscopic images without demographic information or additional radiological findings analysis. The three CNN classifiers achieved an AUC of 0.8 or more, and the DenseNet model predicted degeneration and non-degeneration with the accuracy of 91% and an AUC of 0.92. The test shows that arthroscopic images are useful for predicting prognosis without the need to combine with other factors.

Meanwhile, according to the present disclosure, prediction is performed under a predetermined purpose using a model implemented in an artificial neural network method, and the artificial neural network will be described below.

A model in the present specification may refer to any type of computer program operating based on a network function, an artificial neural network and/or a neural network. In the present specification, a model, a neural network, a network function, and a neural network may be used as an interchangeable meaning. In the neural network, at least one node is interconnected through at least one link to form the relationship of an input node and an output node relationship. The characteristics of the neural network may be determined depending on the number of nodes and links in the neural network, the relationship between the nodes and the links, and the value of a weight assigned to each of the links. The neural network may include at least one set of nodes. A subset of nodes constituting the neural network may form a layer.

A deep neural network (DNN) may refer to a neural network including a plurality of hidden layers in addition to an input layer and an output layer, and as illustrated in FIG. 4 showing the concept of the DNN, at least one hidden layer, preferably, at least two hidden layers are provided in the middle of the DNN.

The DNN may include a convolutional neural network (CNN), a vision transformer, a recurrent neural network (RNN), a long short term memory (LSTM) network, a generative pre-trained transformer (GPT), an auto encoder, a generative adversarial networks (GAN), a restricted Boltzmann machines (RBM), a deep trust network (DBN), a Q network, a U network, a Sham network, a Generative Adversarial Network (GANs), and a Transformer.

Alternatively, according to an embodiment, the DNN may be a model trained in a transfer learning scheme. In this case, the transfer learning scheme is to obtain a pre-trained model (or a base part) having a first task through a scheme as a MLM or a NNSP by pre-training a larger capacity of learning data, which is not labeled, through a semi-supervised or self-learning scheme, and to implement a target model by training the labeled learning data through the supervised scheme to perform a fine-tuning operation, such that the pre-trained model is appropriate to the second task. A Bidirectional Encoder Representations from Transformers (BERT) model is one of models trained through such a transfer learning scheme, but the present disclosure is not limited thereto.

The description of the above-described DNN is provided only for the illustrative purpose, and the present disclosure is not limited thereto. In this case, the CNN described above includes a feature extracting part (or feature learning part) which extracts features from an image, and a classification part which performs classification using the extracted features. The feature learning part may include a convolutional layer to extract features using a kernel from an image, a ReLU layer that is one of an activation function, and a pooling layer to reduce the dimension of data, but the present disclosure is not limited thereto. In addition, the classification part may include a flatten layer which lines up the features extracted by the feature extracting part, and a fully connected layer and a softmax function that substantially performs the classification, but the present disclosure is not limited thereto.

The neural network may be trained in at least one of supervised learning, unsupervised learning, semi-supervised learning, self-supervised learning, or reinforcement learning. The training of the neural network may be a process of applying knowledge for the neural network to perform a specific operation, to the neural network.

The neural network may be trained to minimize the error of an output. In training of the neural network, learning data is repeatedly input to the neural network, an error of the output and target of the neural network for the learning data is calculated, and the error of the neural network is back-propagated from the output layer of the neural network to the input layer to reduce the error and to update the weight of each node of the neural network. For supervised learning, data (labeled data) labeled with a correct answer may be used for each learning data. For unsupervised learning, unlabeled data having no label of the correct answer may be used for each learning data. The amount of change in a connection weight of each updated node may be determined depending on a learning rate. The calculation of the neural network for the input data and the back-propagation of the error may constitute a learning cycle (epoch). The learning rate may be applied differently depending on the number of repetitions of the learning cycle of the neural network. Schemes, such as increasing learning data, regularization, dropout that deactivates some nodes, and a batch normalization layer, may be applied to prevent over-fitting.

Meanwhile, the model disclosed according to an embodiment may borrow at least a portion of the transformer. The transformer may include an encoder which encodes embedded data and a decoder that decodes encoded data. The transformer may have a structure which receives a series of data and outputs different types of data through encoding and decoding steps. According to an embodiment, a series of data may be processed in the form allowing the computation by the transformer. The process of processing the series of data into the form allowing the computation by the transformer may include an embedding process. Expressions such as data tokens, embedding vectors, and embedding tokens may refer to embedded data in the form that the transformer may process.

In order for the transformer to encode and decode a series of data, the encoder and the decoder in the transformer may be processed using an attention algorithm. The attention algorithm may refer to an algorithm to obtain a similarity for at least one key related to a given query, reflect the similarity obtained in such a manner to a value corresponding to each key, and calculate an attention value by performing weighted-sum for values obtained reflecting the similarity.

The attention algorithm may be variously classified depending on a manner of setting a query, a key, and a value. For example, when an attention is obtained by setting the query, the key, and the value to the same value, the attention algorithm may be a self-attention algorithm. When the attention is found by finding an individual attention head for each divided embedding vector by reducing the embedding vector to process an input series of data in parallel, the attention algorithm may be a multi-head attention algorithm.

According to an embodiment, the transformer may include modules to perform a plurality of multi-head self-attention algorithms or a plurality of multi-head encoder-decoder algorithms. According to an embodiment, the transformer may also include an additional component, such as embedding, normalization, or softmax, other than the attention algorithm. A manner of configuring the transformer using the attention algorithm may include a manner disclosed in “Vaswani et al., Attention Is All You Need, 2017 NIPS”, which is incorporated into the present specification by reference.

The transformer may be applied to various data domains such as embedded natural language, divided image data, and audio waveforms to convert a series of input data into a series of output data. To convert data having various data domains into a series of data that is able to be input to the transformer, the transformer may embed the data. The transformer may process additional data representing a relative positional or topological relationship between a series of input data. Alternatively, vectors representing the relative positional or topological relationship between a series of input data may be additionally reflected in the series of input data such that the series of input data are embedded. According to an embodiment, the relative positional relationship between the series of input data may include a word order within a natural language sentence, a relative positional relationship of the divided images, and a temporal order of divided audio waveforms, but the present disclosure is not limited thereto. The process of adding information representing the relative positional or topological relationship between the series of input data may be referred to as positional encoding.

The above-described program may include codes in a computer language, such as “C”, “C++”, “JAVA”, or machine language, which is readable through a device interface of the computer by a central processing unit (CPU), to execute, by the computer, the method implemented in program, by reading out the program. The code may include a functional code related to a function for determining functions necessary for executing the methods, and may include a control code related to execution procedures necessary for executing the functions in specific sequence through the processor of the computer. In addition, such a code may include additional information necessary to execute the functions through the processor of the computer, or a code related to memory reference regarding a position (an address) of an internal memory or an external memory of the computer, to which media are referred. In addition, when the processor of the computer needs to communicate with a second computer or a second server in a remote place to execute the functions, the code may include a code related to communication, in relation to, for example, a manner to communicate with the second computer or the second server provided in the remote place through the communication module of the computer or the type or a media to be transmitted or received in communication.

The storage medium refers to a medium for permanently storing data and readable by machine, as well as a medium, such as a register, a cache, or a memory, within a short period time. The storage medium may be a read-only memory (ROM), a random-access memory (RAM), a compact disk read-only memory (CD-ROM), a magnetic tape, a floppy disk, an optical data storage device, but the present disclosure is not limited thereto. In other words, the program may be stored in various recording media on various servers to be accessed by the computer, or various recording media on the computer of the user. In addition, the medium may store a code readable by the computer in a distribution scheme, as computer systems are distributed over a network.

The method or the algorithm steps described regarding the embodiment of the present disclosure may be implemented in hardware, and implemented with a software module executed by the hardware, or the combination of the software and the hardware. A soft module may reside in a random access memory (RAM), a read only memory (ROM), an erasable programmable ROM (EPROM), an electrically erasable programmable ROM (EEPROM), a flash memory, a hard disk, a removable disk, a CD-ROM, or a computer readable recording medium well known in the art to which the present disclosure pertains.

According to an embodiment of the present disclosure, the model may be pre-trained based on the deep-learning algorithm using the arthroscopic image appropriate to determining the quality of the tendon, and the probability of the re-rupture for the surgical site of the relevant patient may be easily predicted, such that the medical staff or the medical institution performs the operation based on the probability of the re-rupture when operating the relevant patient. Accordingly, the probability of the re-rupture may be lowered. In particular, the re-rupture of a site for the entering of suture may be predicted in real time based on the tendon quality, thereby enhancing the suture technique during the operation and reducing the re-rupture thereafter. In addition, when the re-rupture is highly predicted even after the site is stitched up, the stitch may be an evidence for an additional skill such as adding acellular demarcation matrix

Meanwhile, according to the present disclosure, the prediction information, which is generated as the probability of the re-rupture is predicted based on the arthroscopic image of the relevant patient after operation, may be transmitted to at least one relevant terminal preset (registered), such that the relevant patient is thoroughly cared after operation.

Effects to be solved by the present disclosure are not limited to the effects mentioned above, and other effects not mentioned will be clearly understood by those skilled in the art from the following description.

Although the embodiment of the present disclosure have been described with reference to accompanying drawings, those skilled in the art should understand that various modifications are possible without departing from the technical scope of the present disclosure or without changing the technical sprite or the subject matter of the present disclosure. Therefore, those skilled in the art should understand that the technical embodiments are provided for the illustrative purpose in all aspects and the present disclosure is not limited thereto.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. An apparatus for predicting re-rupture of a tendon based an artificial intelligence (AI), the apparatus comprising:
a communication module configured to make communication with an external device;
an acquiring module configured to acquire at least one arthroscopic image including a surgical portion of a patient experiencing a surgery;
a storage module configured to store at least one process based on the AI;
a control module configured to perform an operation for predicting the re-rupture of the tendon based on the AI, through the at least one process, and
wherein the control module is configured to:
perform a pre-processing operation for the at least one arthroscopic image, predict a probability of the re-rupture of the tendon by inputting the at least one arthroscopic image, which is pre-processed, into a pre-trained model based on the AI, and generate prediction information for the patient based on a prediction result.

2. The apparatus of claim 1, further comprising:
a training module configured to:
collect and pre-process a plurality of arthroscopic images for each of different patients for a preset period, and train an original model by inputting the plurality of arthroscopic images, which are preprocessed, as learning data to implement the pre-trained model.

3. The apparatus of claim 2, wherein the plurality of arthroscopic images are taken and collected with respect to a relevant patient at mutually different time points, for the preset period, and include a time-series change in a surgical site of the relevant patient.

4. The apparatus of claim 3, wherein the training module classifies, manages, and uses the plurality of arthroscopic images into a first group for a patient having no re-rupture and a second group having re-rupture.

5. The apparatus of claim 4, wherein the training module specifies at least one region in each of the plurality of arthroscopic images when pre-processing the plurality of arthroscopic images, and labels at least one of whether the at least one region specified is re-ruptured or a time point at which the at least one region specified is re-ruptured.

6. The apparatus of claim 5, wherein the prediction information includes:
a region to be predicted to be re-ruptured with respect to the patient, a tendon state for each region, a probability of the re-rupture, and timing predicted to be re-ruptured.

7. The apparatus of claim 6, wherein the control module performs a categorizing operation depending on the probability of the re-rupture of the patient.

8. The apparatus of claim 2, wherein the training module removes at least one layer from the original model, finely adjusts a parameter using the additional layer, and performs the training in a preset number of times using average square root propagation (RMSProp) at a preset speed.

9. The apparatus of claim 8, wherein the training module evaluates and verifies performance by using predictive accuracy, F1 score, AUC, sensitivity, and specificity with respect to the pre-trained model.

10. The apparatus of claim 9, wherein the training module uses Equation 1 to calculate the predictive accuracy and the FI score and uses a J statistics to calculate a threshold value for the sensitivity and a threshold value for the specificity,
$$Predictive accuracy = \frac{TP + TN}{TP + FP + FN + TN} \qquad \underline{Equation 1}$$
$$Positive predictive value = \frac{TP}{TP + FP}$$
$$Negative predictive value = \frac{TN}{TN + FN}$$
$$F 1 score = 2 \times \frac{( Senstivity \times Positive predictive value )}{( Senstivity + Positive predictive value} ,$$
in which ‘TP’ denotes ‘true positive’, ‘TN’ denotes ‘true negative’, ‘FP’ denotes ‘false positive’, and ‘FN’ denotes ‘false negative’.

11. A method for predicting re-rupture of a tendon based on artificial intelligence (AI), which is performed by an apparatus, the method comprising:
acquiring at least one arthroscopic image including a surgical site of a patient experiencing a surgery;
performing a pre-processing operation for the at least one arthroscopic image;
predicting a probability of the re-rupture of the tendon by inputting the at least one arthroscopic image, which is pre-processed, into a pre-trained model based on the AI; and
generating prediction information for the patient based on a prediction result.

12. The method of claim 11, further comprising:
collecting a plurality of arthroscopic images for each of different patients for a preset period;
performing a pre-processing operation for the plurality of arthroscopic images; and
training an original model by inputting the plurality of arthroscopic images, which are preprocessed, as learning data to implement the pre-trained model.

13. The method of claim 12, wherein the plurality of arthroscopic images are taken and collected with respect to a relevant patient at mutually different time points for the preset period, and include a time-series change in a surgical site of the relevant patient.

14. The method of claim 13, wherein the collecting of the plurality of arthroscopic images includes:
classifying, managing, and using the plurality of arthroscopic images into a first group for a patient having no re-rupture and a second group having re-rupture.

15. The method of claim 14, wherein the pre-processing includes:
specifying at least one region in each of the plurality of arthroscopic images when pre-processing the plurality of arthroscopic images, and labeling at least one of whether the at least one region specified is re-ruptured or a time point at which the at least one region specified is re-ruptured.

16. The method of claim 15, wherein the prediction information includes:
a region to be predicted to be re-ruptured with respect to the patient, a tendon state for each region, a probability of the re-rupture, and timing predicted to be re-ruptured.

17. The method of claim 16, wherein the generating of the prediction information to be provided to a terminal of a medicine staff includes:
performing a categorizing operation depending on the probability of the re-rupture of the patient.

18. The method of claim 12, wherein the implementing of the pre-trained model includes:
removing at least one layer from the original model, finely adjusting a parameter using the additional layer, and performing the training operation in a preset number of times using average square root propagation (RMSProp) at a preset speed.

19. The method of claim 18, wherein the implementing of the pre-trained model includes:
evaluating performance by using predictive accuracy, F1 score, AUC, sensitivity, and specificity with respect to the pre-trained model.

20. The method of claim 19, wherein the implementing of the pre-trained model includes:
using following Equation 1 to calculate the predictive accuracy and the FI score and using a J statistics to calculate a threshold value for the sensitivity and a threshold value for the specificity,
$$Predictive accuracy = \frac{TP + TN}{TP + FP + FN + TN} , \qquad \underline{Equation 1}$$
$$Positive predictive value = \frac{TP}{TP + FP}$$
$$Negative predictive value = \frac{TN}{TN + FN}$$
$$F 1 score = 2 \times \frac{( Senstivity \times Positive predictive value )}{( Senstivity + Positive predictive value}$$
in which ‘TP’ denotes ‘true positive’, ‘TN’ denotes ‘true negative’, ‘FP’ denotes ‘false positive’, and ‘FN’ denotes ‘false negative’.
